(19) United States
(12) Patent Application Publication
(10) Pub. No.: US 2007/0243146 A1
(43) Pub. Date: Oct. 18, 2007

(54) Vitamin K1 as Energizer in Cosmetic Formulations

(76) Inventor: Jochen Klock, Freiburg (DE)

Correspondence Address: NIXON & VANDERHYE, PC, 901 NORTH GLEBE ROAD, 11TH FLOOR, ARLINGTON, VA, 22203, US

(21) Appl. No.: 11/629,206

(22) PCT Filed: Jun. 16, 2005
(86) PCT No.: PCT/EP05/06478

§ 371 Date: Jan. 3, 2007

(30) Foreign Application Priority Data
Jun. 18, 2004 (EP) 04014378.6

(51) Int. Cl. A61Q 19/00; A61K 8/30; A61K 8/35; A61K 8/67

(52) U.S. Cl. 424/59; 424/69; 424/70.1

(57) ABSTRACT

The invention relates to the use of vitamin K₁ as energizer in cosmetic formulations and to specific cosmetic formulations containing vitamin K₁.

The present invention relates to the use of vitamin K₁ as energizer in cosmetic formulations and to specific cosmetic formulations containing vitamin K₁.

Vitamin K is a general name for a group of compounds containing a 2-methyl-1,4-naphthoquinone nucleus with different lipophilic side chains at position 3, in the following also referred to as “the K-vitamins”. Vitamin K naturally occurs in the form of vitamin K₁ (phytomenadione, phytonadione, α-phylloquinone) which is produced by green leafy vegetables and vitamin K₂ (menaquinone) which is produced by gastrointestinal bacteria in basically two different sub-types, namely vitamin K₂₍₃₅₎ (C₄₆H₆₄O₂) and vitamin K₂₍₃₀₎ (C₄₁H₅₆O₂). Vitamin K₃ (menadione) is a pro-vitamin which can be converted to vitamin K₂ by animals.

The chemical structure of vitamins K₁, K₂₍₃₅₎, K₂₍₃₀₎ and K₃ are depicted here below:

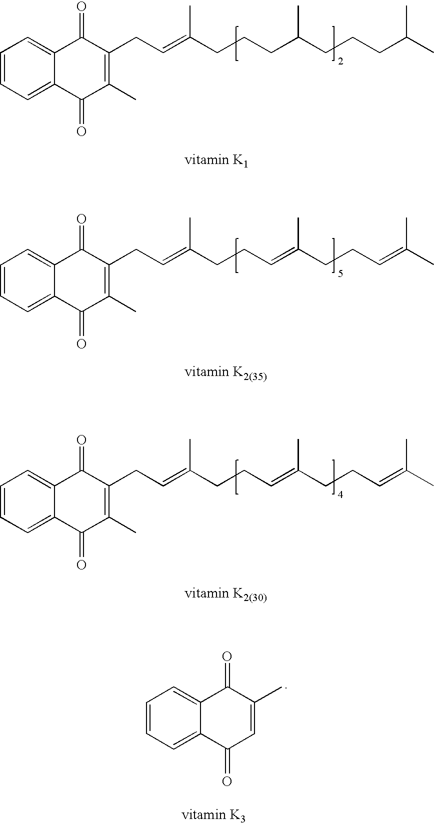

Besides the naturally occurring vitamins K₁, K₂₍₃₅₎, K₂₍₃₀₎ and K₃ artificial analogues have been synthesized including K₄, K₅, K₆ and K₇. Menadione and menaquinones may occasionally have toxic effects in high doses whereas phylloquinone seems to be safe even in massive overdoses. Phylloquinone is therefore the preferred form of the vitamin for human use and the only approved one for cosmetic use.

The best known role for the K-vitamins in humans is as co-factor for the synthesis of six of the proteins involved in blood clotting. These proteins are inactive proenzymes which are converted to active enzymes in the presence of calcium during the coagulation process. These proteins contain an unusual amino acid, γ-carboxy-glutamate. This is formed by the carboxylation of glutamic acid residues in the protein by the enzyme γ-glutamyl carboxylase, in a vitamin K dependent reaction.

Besides blood clotting, there is evidence that K-vitamins can have clinically relevant effects on bone. In women with osteoporosis, a controlled study showed that 45 mg/day of vitamin K₂ could reduce the risk of bone fractures and slow down but not prevent a progressive loss of bone mineral density (M. Shiraki et al., J. Bone Mineral Res. 2000, 15, 515-21).

It is also known that the vitamin K₃ (menadione) may partially restore a deficiency of complex I in the respiratory chain located in the inner mitochondrial membrane. Complex I mediates the electron transfer from NADH to coenzyme Q. There is experimental evidence that menadione can be reduced by the enzyme DT-diaphorase and that the reduced menadione itself may reduce coenzyme Q thereby surpassing the block at complex I (F. A. Wijburg et al., Biochem. Int. 1990, 22, 303-9). Contrary thereto, when coenzyme Q₁₀ and vitamin K₁ are tested on eukaryotic cell lines to activate transplasma membrane electron transport in order to stimulate the cell growth, it is found that only coenzyme Q₁₀ is able to stimulate these cells whereas vitamin K₁ shows no efficacy (I. L. Sun et al., Protoplasma 1995, 184, 214-9).

Pharmaceutical compositions comprising vitamin K₁ are known in the prior art. Such compositions are usually intended for applications in connection with the known blood coagulation activity of vitamin K₁. In this regard it can be referred to e.g. WO 95/11015, WO 97/39746 and US 2002/0025983.

The use of K-vitamins for cosmetic purposes is also known in the prior art. In this regard it can be referred to e.g. DE-A 100 03 786 and US-A 2003/0170187. DE-A 100 03 786 discloses cosmetic or dermatological formulations containing as an active ingredient vitamin K₁ and one or more antioxidants and/or one or more UV-filters. The formulations are said to stabilize the active ingredient contained therein. US-A 2003/0170187 discloses the use of nano-sized vitamin K for the improvement of various aesthetic aspects of the skin including reduction of the reddened, black and/or blue appearance of the skin. A topical gel is disclosed containing vitamin K and vitamin C which can be used around the eyes, arms and legs to effectively and quickly reduce the discoloration of the skin and accelerate healing. In another embodiment, a topical cream is disclosed for use on dark circles or splotches under the eye. With regular use it is said to improve the aesthetic aspects and provides a more youthful look. Its particular properties allow reflection of light which minimizes the transparency of the skin under the eye. Also cosmetic purposes are basically going back to the known blood coagulation activity of vitamin K1.

However, it is unknown that vitamin K₁ exhibits an energizing effect when used in cosmetic formulations.

It is known from the prior art that certain other ingredients of cosmetic formulations may act as energizers. For example, US 2003/0191946 discloses cosmetic and perfume products containing amber. An additional cosmetic effect is said to be achieved by adding energizing or active substances as sea algae, yeast extracts, enzymes, liposomes, vitamins and others to modified amber. WO 95/31177 discloses a cosmetic or pharmaceutical composition containing an effective amount of laminarin or laminarin-derived oligosaccharides. The compositions are said to have stimulating, regenerating, conditioning and energizing effects on human dermis fibroblasts and human epidermis keratinocytes. FR-A 2629007 discloses cosmetics comprising DL- or L-carnitine in free form or as salts with Krebs cycle acids. Since carnitine has a lipolytic activity, the cosmetics are said to induce weight loss and have an energizing, stimulating and tonic effect.

The energizing effect of the cosmetic formulations known from the prior art is not satisfactory in every respect. A good energizer should improve the condition of the skin including the head skin, the hair and the nail plates. However, most prior art formulations only show a weak energizing effect. Furthermore, some energizers are difficult to formulate, not almost very compatible with other ingredients, unstable in the formulation and do not or only slowly penetrate through the skin or may cause skin irritations, allergies, and the like.

Therefore, there is a need for cosmetic formulations containing energizers which overcome the drawbacks of the formulations disclosed in the prior art.

Thus, it is an object of the present invention to provide energizers which have an energizing effect which is comparable, preferably better than the energizing effect of the formulations of the prior art. A further object of the present invention is to provide novel cosmetic formulations with excellent cosmetic properties which in addition have an energizing effect.

This technical problem can be solved by the subject matter of the claims.

It has been surprisingly found that vitamin K₁ exhibits an energizing effect when used in cosmetic formulations. Experiments have revealed that vitamin K₁ is suitable to vitalize normal human skin by increasing the energy level of the skin cells.

For the purpose of the specification an energizer is a compound exhibiting an energizing effect when contained in cosmetic formulations. The energizer may have a stimulating, regenerating or conditioning effect on skin cells which can be correlated with an increase of the bioactivity of the cells, such as a stimulation of the neosynthesis of proteins. Preferably, however, the effect is correlated with an increase of the energy level of the cells. For example, the increase of the energy level can be monitored by measuring the ATP concentration in skin cells. The person skilled in the art is aware of suitable methods for measuring energy levels of cells, such as ATP levels in vitro and in vivo. A method is additionally disclosed in the examples.

The increased energy level of the skin cells has a vitalizing cosmetic effect such that the skin condition is improved, i.e. the skin becomes properly tight; silky smooth and nice in touch and appears healthy, visibly younger and fresh having a delicate radiance. Furthermore, the condition of the head skin is improved and the hair bulbs are strengthened thereby preventing hair loss. An energizing action on scalp and hair roots promotes vitality and intensed shine. Hair becomes fluffy and glossy and regains its vitality and health; the nail plates are strengthened. Grey and pale skin is improved by a vitalizing effect and skin is nourished. The increased energy level sharpens up stressed and tired skin, and stimulates its natural defenses.

The inventors of the present invention have found that vitamin K₁ may be used as energizer in a cosmetic formulation. Preferably, the content of vitamin K₁ in the cosmetic formulation is within the range of 0.01 to 5.00 wt.-%, more preferably 0.05 to 2.00 wt.-%.

Preferably, vitamin K₁ is used in the cosmetic formulation in a concentration effective to provide a concentration of vitamin K₁ in skin cells above 1 μM, preferably above 10 μm, more preferably between 10 and 50 μM, in particular between 10 and 25 μM, most preferably between 15 and 25 μm, when the cosmetic formulation is applied to the skin cells. The concentration of vitamin K₁ in the cosmetic formulation required to provide the respective concentration in the skin cells may be determined by routine experimentation. The person skilled in the art is aware of methods to measure the concentration of vitamin K₁ in skin cells. In this regard it can be referred to e.g. (Kamali F. et al, Am J Hematol 2001, 68(3), 159-63).

The present invention also relates to specific cosmetic formulations containing vitamin K₁.

The term “cosmetic formulation” as used in the present application refers to compositions as defined under the heading “Kosmetika” in Römpp Lexikon Chemie, 10th edition 1997, Georg Thieme Verlag Stuttgart, New York.

In a preferred embodiment the present invention relates to a cosmetic formulation containing vitamin K₁ and vitamin C or a derivative of vitamin C. Preferably, the formulation contains vitamin K₁ and a derivative of vitamin C, more preferably a compound selected from the group consisting of ascorbyl phosphate, ascorbyl acetate, ascorbyl palmitate, ascorbyl tetraisopalmitate, ascorbyl glucoside and a cosmetically acceptable salt thereof, particularly trisodium ascorbyl phosphate which is commercialized e.g. as STAY-C® 50.

The compositions of the present invention can contain usual cosmetically acceptable excipients or diluents. If nothing else is stated, the excipients, additives, diluents, etc. mentioned in the following are suitable for cosmetic compositions.

If nothing else is stated, in this application parts and percentages are per weight and are based on the weight of the composition.

Preferably, the compositions are topical compositions, such as liquid or solid oil-in-water emulsions, water-in-oil emulsions, multiple emulsions, microemulsions, PET-emulsions, bickering emulsions, hydrogels, alcoholic gels, lipogels, one or multiphase solutions, foams, ointments, plasters, suspensions, powders, crémes, cleanser, soaps and other usual compositions, which can also be applied by pens, as masks or as sprays.

The compositions can also contain usual cosmetic adjuvants and additives, such as preservatives/antioxidants, fatty substances/oils, water, organic solvents, silicones, thickeners, softeners, emulsifiers, sunscreens, cosmetic actives antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, e.g. those suited for providing a photoprotective effect by physically blocking out ultraviolet radiation, or any other ingredients usually formulated into cosmetics. A good overview of suitable additives for cosmetic compositions can also be found e.g. in WO 03/082232. The additives disclosed in this document, in particular the waxes, thickeners, structuring agents, film forming agents and conditioning ingredients are also suitable for the compositions and included herein by reference. Of course, the stabilizing compositions disclosed in this document can also be used for preparing the compositions.

The composition can also contain one or more additional pharmaceutically or cosmetically active ingredients, in particular for preventing or reducing acne, wrinkles, lines, atrophy, inflammation, as well as topical anesthetics, artificial tanning agents and accelerators, antimicrobial agents, and antifungal agents and sunscreening additives.

Examples are peptides (e.g., Matrixyl™ [pentapeptide derivative]), farnesol, bisabolol, phytantriol, glycerol, urea, guanidine (e.g., amino guanidine); vitamins and derivatives thereof such as ascorbic acid, vitamin A (e.g., retinoid derivatives such as retinyl palmitate or retinyl propionate), vitamin E (e.g., tocopherol acetate), vitamin B₃ (e.g., niacinamide) and vitamin B₅ (e.g., panthenol) and the like and mixtures thereof, wax-based synthetic peptides (e.g., octyl palmitate and tribehenin and sorbitan isostearate and palmitoyl-oligopeptide), anti-acne medicaments (resorcinol, salicylic acid, and the like); antioxidants (e.g., phytosterols, lipoic acid); flavonoids (e.g., isoflavones, phytoestrogens); skin soothing and healing agents such as aloe vera extract, allantoin and the like; chelators and sequestrants; and agents suitable for aesthetic purposes such as essential oils, fragrances, skin sensates, opacifiers, aromatic compounds (e.g., clove oil, menthol, camphor, eucalyptus oil, and eugenol), desquamatory actives, anti-acne actives, vitamin B₃ compounds, anti-oxidants, peptides, hydroxy acids, radical scavengers, chelators, farnesol, anti-inflammatory agents, topical anesthetics, tanning actives, skin lightening agents, anti-cellulite agents, flavonoids, antimicrobial actives, and antifungal actives, in particular bisabolol, alkyldiols such as 1,2-pentandiol, hexanediol or 1,2-octanediol, vitamins, panthenol, phytol, phytantriol, ceramides and pseudoceramides, amino acids and bioactive peptides, protein hydrolysates, AHA acids, polyunsaturated fatty acids, plant extracts, DNA or RNA and their fragmentation products or carbohydrates, biotin, lipoic acid, conjugated fatty acids, carnitin, vitamin E, A, C, B3, B6, B12, panthenol, phytantriol, oligopeptides, carnosin, biochinonen, phytofluen, phytoen, folic acid, and their corresponding derivatives.

In a preferred embodiment the present invention relates to a cosmetic formulation containing vitamin K₁ and an UV absorbing substance. Suitable UV absorbing substances are UV-A, UV-B filters and broad spectrum screening agents. Examples of UV-B or broad spectrum screening agents, i.e. substances having absorption maximums between about 290 and 340 nm, which are preferred for combination with vitamin K1, are the following organic and inorganic compounds:

Compounds belonging to the groups of acrylates, p-aminobenzoates, camphor derivatives (such as of benzylidene camphor type), cinnamates, benzophenones, esters of benzalmalonic acid, esters of 2-(4-ethoxy anilinomethylene)propandioic, imidazole derivatives, salicylates, triazone derivatives, triazol derivatives, dibenzoylmethanes, amino substituted hydroxybenzophenones, phenyl-benzimidazoles, anthranilates, phenyl-benzoxazoles, 1,4-dihydropyranes, organosiloxane compounds and others representing state of the art and known to those skilled in the art to be highly active.

Examples for acrylates include 2-ethylhexyl 2-cyano-3,3-diphenylacrylate (octocrylene, PARSOL® 340) and ethyl 2-cyano-3,3-diphenylacrylate.

Examples for p-aminobenzoates include 4-amino benzoic acid, 4-aminobenzoic acid-2,3-dihydroxypropylester, 4-(bis(2-hydroxypropyl)amino)benzoic acid ethyl ester, 4-(dimethyl-amino)benzoic acid-2-ethylhexylester (e.g. Eusolex® 6007) and ethoxylated 4-aminobenzoic acid ethyl ester (e.g. Uvinul® P25), N-oxypropylenated ethyl p-aminobenzoate and glyceryl p-aminobenzoate.

Examples for camphor derivatives include 4-methyl benzylidene camphor (PARSOL® 5000), 3-benzylidene camphor, camphor benzalkonium methosulfate, polyacrylamidomethyl benzylidene camphor, sulfo benzylidene camphor, sulphomethyl benzylidene camphor and therephthalidene dicamphor sulfonic acid.

Examples for cinnamates include octyl methoxycinnamate (PARSOL® MCX), ethoxyethyl methoxycinnamate, diethanolamine methoxycinnamate (PARSOL® Hydro), isoamyl methoxycinnamate and cinnamic acid derivatives bound to siloxanes.

Examples for benzophenones include benzophenone-3, benzophenone-4,2,2′,4,4′-tetra-hydroxy-benzophenone and 2,2′-dihydroxy-4,4′dimethoxybenzophenone.

Examples for esters of benzalmalonic acid include di(2-ethylhexyl) 4-methoxybenzal-malonate.

Examples for esters of 2-(4-ethoxy anilinomethylene)propandioic acid include 2-(4-ethoxy anilinomethylene)propandioic acid diethyl ester as described in EP-A 895 776.

Examples of organosiloxane compounds are organosiloxane compounds containing benzmalonate groups as described in the European Patent Publications EP 0358584 B1, EP 0538431 B1 and EP 0709080 A1, in particular Parsol SLX.

Further examples of useful UV-screening agents are drometrizole trisiloxane (Mexoryl XL) and pigments such as microparticulated TiO₂, and the like. The term “microparticulated” refers to a particle size from about 5 nm to about 200 nm, particularly from about 15 nm to about 100 nm. The TiO₂ particles may also be coated by metal oxides such as e.g. aluminum or zirconium oxides or by organic coatings such as e.g. polyols, methicone, aluminum stearate, alkyl silane. Such coatings are well known in the art.

Examples for imidazole derivatives include 2-phenyl benzimidazole sulfonic acid and its salts (PARSOL®HS). Salts of 2-phenyl benzimidazole sulfonic acid are e.g. alkali salts such as sodium- or potassium salts, ammonium salts, morpholine salts, salts of primary, sec. and tert. amines like monoethanolamine salts and diethanolamine salts.

Examples for salicylate derivatives include isopropylbenzyl salicylate, benzyl salicylate, butyl salicylate, octyl salicylate (NEO HELIOPAN OS), isooctyl salicylate or homomethyl salicylate (homosalate, HELIOPAN).

Examples for triazone or triazine derivatives include octyl triazone (UVINUL T-150), dioctyl butamido triazone (UVASORB HEB) and ethoxy phenol methoxy phenyl triazine (Tinosorb S).

Examples for triazol derivatives include benzotriazoles such as 2-(2-hydroxy-5-methylphanyl)benzotriazol, 2,2′-methylene-bis-(6-(2H-benzotriazole-2-yl)-4-(1,1,3,3,-tetra-methylbutyl)-phenol (TINOSORB M) as well as triazols described in EP-A 893 119.

Examples for dibenzoylmethane derivatives include compounds such as 4-tert. butyl-4′-methoxydibenzoyl-methane (PARSOL® 1789), dimethoxydibenzoylmethane and isopropyl-dibenzoylmethane.

Examples for amino substituted hydroxybenzophenones include compounds such as 2-(4-Diethylamino-2-hydroxy-benzoyl)-benzoic acid hexyl ester ester as described in EP-A 1 046 391.

Examples for phenylene-1,4-bis-benzimidazolsulfonic acids or salts include 2,2-(1,4-phenylene)bis-(1H-benzimidazol-4,6-disulfonic acid) (Neoheliopan AP).

As dibenzoylmethane derivatives have limited photostability it may be desirable to photostabilize these UV-A screening agents. Thus, the term “conventional UV-A screening agent” also refers to dibenzoylmethane derivatives such as e.g. PARSOL® 1789 stabilized by, e.g.,
3,3-Diphenylacrylate derivatives as described in the European Patent Publications EP-A 0 514 491 and EP-A 0 780 119;
Benzylidene camphor derivatives as described in the U.S. Pat. No. 5,605,680;
Organosiloxanes containing benzmalonate groups as described in the European Patent Publications EP-A 0358584, EP-A 0538431 and EP-A 0709080, such as Parsol SLX.

A good overview of UV-A and UV-B-filters which can be added to the compositions of the present invention can also be found in DE-A 103 27 432. All UV-filter compounds disclosed in this document are also useful as components for the compositions of the present invention and are included herein by reference.

In another preferred embodiment the present invention relates to a cosmetic formulation containing vitamin K₁ and an UV absorbing substance selected from the group consisting of
polysiloxane-based UV absorbers, such as α-(trimethylsilyl)-ω)-(trimethylsilyloxy)-poly[oxy(dimethyl)silylene]-co-[oxy(methyl)(2-{p-[2,2-bis(ethoxycarbonyl)vinyl]-phenoxy}-1-methylene-ethyl)silylene]-co-[oxy(methyl)-(2-{p-[2,2-bis(ethoxycarbonyl)-vinyl]phenoxy}prop-1-enyl)silylene] (Parsol® SLX),
1-(4-tert-butylphenyl)-3-(4-methoxyphenyl)-propane-1,3-dione (Parsol® 1789),
2-cyano-3,3-diphenyl-acrylic acid 2-ethyl-hexyl ester (Parsol® 340),
(E)-rac-1,7,7-trimethyl-3-(4-methyl-benzylidene)-bicyclo[2.2.1]heptan-2-one (Parsol® 5000),
2-phenyl-1H-benzimidazole-5-sulphonic acid (Parsol® HS), and
(E)-3-(4-methoxy-phenyl)-propionic acid 2-ethyl-hexyl ester (Parsol® MCX).

In still another preferred embodiment the present invention relates to a cosmetic formulation containing vitamin K₁ and coenzyme Q₁₀, optionally in combination with vitamin C or a derivative thereof as described above.

In a preferred embodiment the cosmetic formulation contains one or more UV absorbing substances as defined above in an amount of from 0.001 to 50 wt.-%, more preferably from 0.1 to 20 wt.-%, still more preferably from 0.2 to 15 wt.-%, most preferably from 0.5 to 10 wt.-% and in particular from 0.75 to 5.25 wt.-%.

In another preferred embodiment the cosmetic formulation contains vitamin C or trisodium ascorbyl phosphate in an amount of from 0.001 to 20 wt.-%, more preferably from 0.01 to 10 wt.-% and most preferably from 0.1 to 3 wt.-%. In still another preferred embodiment the cosmetic formulation contains coenzyme Q₁₀ in an amount of from 0.001 to 1 wt.-%, more preferably from 0.01 to 0.3 wt.-%.

Preferably, the cosmetic formulation according to the invention is a topical formulation, more preferably a topical formulation selected from the group consisting of creams, pastes, sticks, cleansers, balms, tonics, fluids, shampoos, hair sprays, conditioners, masks, powders, enamels, enamel removers, lipsticks, foams, oils, soaps, peelings, serums, ointments, gels, lotions, liquids and facial tissues.

The cosmetic formulations of the present invention contain vitamin K₁ with cosmetically acceptable excipients or diluents.

If nothing else is stated, in this application parts and percentages are per weight (wt.-%) and are based on the weight of the formulation.

Preferably, the cosmetic formulation of the present invention contains vitamin K₁ in a concentration of 0.001 to 50 wt.-%, more preferably 0.01 to 5.00 wt.-%, most preferably 0.05 to 2.00 wt.-% based on the weight of the formulation.

Preferably, the cosmetic formulations of the present invention are topical formulations, such as liquid or solid oil-in-water emulsions, water-in-oil emulsions, multiple emulsions, microemulsions, PET-emulsions, bickering emulsions, hydrogels, alcoholic gels, lipogels, one or multiphase solutions, foams, ointments, plasters, suspensions, powders, cremes, cleanser, soaps and other usual formulations, which can also be applied by pens, as masks or as sprays.

The cosmetic formulations of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, e.g. those suited for providing a photoprotective effect by physically blocking out ultraviolet radiation, or any other ingredients usually formulated into cosmetics or medicaments.

An additional amount of antioxidants/preservatives is generally preferred Based on the invention all known antioxidants usually formulated into cosmetics or medicaments can be used. Especially preferred are antioxidants chosen from the group consisting of amino acids (e.g. glycine, histidine, tyrosine, tryptophane) and their derivatives, imidazole (e.g. urocanic acid) and derivatives, peptides such as D,L-carnosine, D-carnosine, L-carnosine and derivatives (e.g. anserine), carotenoids, carotenes (e.g. α-carotene, β-carotene, lycopene) and derivatives, chlorogenic acid and derivatives, lipoic acid and derivatives (e.g. dihydrolipoic acid), aurothioglucose, propylthiouracil and other thiols (e.g. thioredoxine, glutathione, cysteine, cystine, cystamine and its glycosyl-, N-acetyl-, methyl-, ethyl-, propyl-, amyl-, butyl- and lauryl-, palmitoyl-; oleyl-, y-linoleyl-, cholesteryl- and glycerylester) and the salts thereof, dilaurylthiodipropionate, distearylthiodipropionate, thiodipropionic acid and its derivatives (ester, ether, peptides, lipids, nucleotides, nucleosides and salts) as well as sulfoximine compounds (such as buthioninsulfoximine, homocysteinsulfoximine, buthioninsulfone, penta-, hexa-, heptathioninsulfoximine) in very low compatible doses (e.g. pmol to μmol/kg), additionally (metal)-chelators (such as α-hydroxyfatty acids, palmic-, phytinic acid, lactoferrin), α-hydroxyacids (such as citric acid, lactic acid, malic acid), huminic acid, gallic acid, gallic extracts, bilirubin, biliverdin, EDTA, EGTA and its derivatives, BHA or BHT, unsaturated fatty acids and their derivatives (such as γ-linoleic acid, linolic acid, oleic acid), folic acid and its derivatives, ubiquinone and ubiquinol and their derivatives, vitamin C and derivatives (such as ascorbylpalmitate and ascorbyltetraisopalmitate, Mg-ascorbylphosphate, Na-ascorbylphosphate, ascorbyl-acetate, ascorbylglycosid), tocopherole and derivates (such as vitamin-E-acetate), mixtures of nat. vitamin E, vitamin A and derivatives (vitamin-A-palmitate and -acetate) as well as coniferylbenzoat, rutinic acid and derivatives, α-glycosylrutin, ferulic acid, furfurylidenglucitol, carnosin, butylhydroxytoluene, butylhydroxyanisole, trihydroxybutyrophenone, urea and its derivatives, mannose and derivatives, zinc and derivatives (e.g. ZnO, ZnSO₄), selen and derivatives (e.g. selenomethionin), stilbenes and derivatives (such as stilbenoxide, trans-stilbenoxide) and suitable derivatives (salts, esters, ethers, sugars, nucleotides, nucleosides, peptides and lipids) of the named active ingredients. One or more preservatives/antioxidants may be present in an amount about 0.01 wt. % to about 10 wt. % of the total weight of the formulation of the present invention. Preferably, one or more preservatives/antioxidants are present in an amount about 0.1 wt. % to about 1 wt. %.

Typically topical cosmetic formulations also contain surface active ingredients like emulsifiers, solubilizers and the like. An emulsifier enables two or more immiscible components to be combined homogeneously. Moreover, the emulsifier acts to stabilize the formulation. Emulsifiers that may be used in the present invention in order to form O/W, W/O, O/W/O or W/O/W emulsions/microemulsions include sorbitan oleate, sorbitan sesquioleate, sorbitan isostearate, sorbitan trioleate, polyglyceryl-3-diisostearate, polyglycerol esters of oleic/isostearic acid, polyglyceryl-6 hexaricinolate, polyglyceryl-4-oleate, polygylceryl-4 oleate/PEG-8 propylene glycol cocoate, oleamide DEA, TEA myristate, TEA stearate, magnesium stearate, sodium stearate, potassium laurate, potassium ricinoleate, sodium cocoate, sodium tallowate, potassium castorate, sodium oleate, and mixtures thereof. Further suitable emulsifiers are phosphate esters and the salts thereof such as cetyl phosphate (Amphisol® A), diethanolamine cetyl phosphate (Amphisol®), potassium cetyl phosphate (Amphisol® K), sodium glyceryl oleate phosphate, hydrogenated vegetable glycerides phosphate and mixtures thereof. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof. The preferred emulsifiers are cetyl phosphate (Amphisol® A), diethanolamine cetyl phosphate (Amphisol®), potassium cetyl phosphate (Amphisol® K), PVP Eicosene copolymer, acrylates/C₁₀₋₃₀-alkyl acrylate crosspolymer, PEG-20 sorbitan isostearate, sorbitan isostearate, and mixtures thereof. The one or more emulsifiers are present in a total amount about 0.01 wt. % to about 20 wt. % of the total weight of the cosmetic formulation of the present invention. Preferably, about 0.1 wt. % to about 10 wt. % of emulsifiers are used.

The lipid phase of the cosmetic formulations can advantageously be chosen from:
mineral oils and mineral waxes;
oils such as triglycerides of caprinic acid or caprylic acid, preferable castor oil;
oils or waxes and other natural or synthetic oils, in an preferred embodiment esters of fatty acids with alcohols e.g. isopropanol, propylene glycol, glycerin or esters of fatty alcohols with carbonic acids or fatty acids;
alkylbenzoates; and/or
silicone oils such as dimethylpolysiloxane, diethylpolysiloxane, diphenyl-polysiloxane, cyclomethicones and mixtures thereof.

Exemplary fatty substances which can be incorporated in the oil phase of the emulsion, microemulsion, oleo gel, hydrodispersion or lipodispersion of the present invention are advantageously chosen from esters of saturated and/or unsaturated, linear or branched alkyl carboxylic acids with 3 to 30 carbon atoms, and saturated and/or unsaturated, linear and/or branched alcohols with 3 to 30 carbon atoms as well as esters of aromatic carboxylic acids and of saturated and/or unsaturated, linear or branched alcohols of 3-30 carbon atoms. Such esters can advantageously be selected from octylpalmitate, octylcocoate, octylisostearate, octyldodecylmyristate, cetearylisononanoate, isopropyl-myristate, isopropylpalmitate, isopropylstearate, isopropyloleate, n-butylstearate, n-hexyllaureate, n-decyloleat, isooctylstearate, isononylstearate, isononylisononanoate, 2-ethyl hexylpalmitate, 2-ethylhexyllaurate, 2-hexyldecylstearate, 2-octyidodecylpalmitate, stearylheptanoate, oleyloleate, oleylerucate, erucyloleate, erucylerucate, tridecylstearate, tridecyltrimellitate, as well as synthetic, half-synthetic or natural mixtures of such esters e.g. jojoba oil.

Other fatty components suitable for use in the cosmetic formulations of the present invention include polar oils such as lecithines and fatty acid triglycerides, namely triglycerol esters of saturated and/or unsaturated, straight or branched carboxylic acid with 8 to 24 carbon atoms, preferably of 12 to 18 carbon-atoms whereas the fatty acid triglycerides are preferably chosen from synthetic, half synthetic or natural oils (e.g. cocoglyceride, olive oil, sun flower oil, soybean oil, peanut oil, rape seed oil, sweet almond oil, palm oil, coconut oil, castor oil, hydrogenated castor oil, wheat oil, grape seed oil, macadamia nut oil and others); apolar oils such as linear and/or branched hydrocarbons and waxes e.g. mineral oils, vaseline (petrolatum); paraffins, squalan and squalene, polyolefins, hydrogenated polyisobutenes and isohexadecanes, favored polyolefins are polydecenes; dialkyl ethers such as dicaprylylether; linear or cyclic silicone oils such as preferably cyclomethicone (octamethylcyclotetrasiloxane; cetyldimethicone, hexamethylcyclotrisiloxane, polydimethylsiloxane, poly(methylphenylsiloxane) and mixtures thereof.

Other fatty components which can advantageously be incorporated in cosmetic formulations of the present invention are isoeikosane; neopentylglycoldiheptanoate; propylen-glycoldicaprylate/dicaprate; caprylic/capric/diglycerylsuccinate; butylenglycol caprylat/caprat; C₁₂₋₁₃-alkyllactate; di-C₁₂₋₁₃ alkyltartrate; triisostearin; dipentaerythrityl hexa-caprylat/hexacaprate; propylenglycolmonoisostearate; tricaprylin; dimethylisosorbid. Especially beneficial is the use of mixtures C₁₂₋₁₅-alkylbenzoate and 2-ethylhexyl-isostearate, mixtures C₁₂₋₁₅-alkylbenzoate and isotridecylisononanoate as well as mixtures of C₁₂₋₁₅-alkylbenzoate, 2-ethylhexylisostearate and isotridecylisononanoate.

The oily phase of the cosmetic formulations of the present invention can also contain natural vegetable or animal waxes such as bee wax, china wax, bumblebee wax and other waxes of insects as well as shea butter and cocoa butter.

A moisturizing agent may be incorporated into a cosmetic formulation of the present invention to maintain hydration or rehydrate the skin. Moisturizers that prevent water from evaporating from the skin by providing a protective coating are called emollients. Additionally an emollient provides a softening or soothing effect on the skin surface and is generally considered safe for topical use. Preferred emollients include mineral oils, lanolin, petrolatum, capric/caprylic triglyceraldehydes, cholesterol, silicones such as dimethicone, cyclometicone, almond oil, jojoba oil, avocado oil, castor oil, sesame oil, sunflower oil, coconut oil and grape seed oil, cocoa butter, olive oil aloe extracts, fatty acids such as oleic and stearic, fatty alcohols such as cetyl and hexadecyl (ENJAY), diisopropyl adipate, hydroxybenzoate esters, benzoic acid esters of C₉₋₁₅-alcohols, isononyl iso-nonanoate, ethers such as polyoxypropylene butyl ethers and polyoxypropylene cetyl ethers, and C₁₂₋₁₅-alkyl benzoates, and mixtures thereof. The most preferred emollients are hydroxybenzoate esters, aloe vera, C₁₂₋₁₅-alkyl benzoates, and mixtures thereof. An emollient is present in an amount of about 1 wt. % to about 20 wt. % of the total weight of the formulation. The preferred amount of emollient is about 2 wt. % to about 15 wt. %, and most preferably about 4 wt. % to about 10 wt. %.

Moisturizers that bind water, thereby retaining it on the skin surface are called humectants. Suitable humectants can be incorporated into a cosmetic formulation of the present invention such as glycerin, polypropylene glycol, 1,2-pentadiol, polyethylene glycol, lactic acid, pyrrolidone carboxylic acid, urea, phopholipids, collagen, elastin, ceramides, lecithin sorbitol, PEG-4, and mixtures thereof. Additional suitable moisturizers are polymeric moisturizers of the family of water soluble and/or swellable and/or with water gelating polysaccharides such as hyaluronic acid, chitosan and/or a fucose rich polysaccharide which is e.g. available as Fucogel®1000 (CAS-Nr. 178463-23-5) by SOLABIA S. One or more humectants are optionally present at about 0.5 wt. % to about 8 wt. % in a cosmetic formulation of the present invention, preferably about 1 wt. % to about 5 wt. %.

The aqueous phase of the preferred topical cosmetic formulation of the present invention can contain the usual cosmetic additives such as alcohols, especially lower alcohols, preferably ethanol and/or isopropanol, low diols or polyols and their ethers, preferably propyleneglycol, glycerin, ethyleneglycol, ethyleneglycol monoethyl- or monobutylether, propyleneglycol monomethyl- or -monoethyl- or -monobutylether, diethyleneglycol mono-methyl- or -monoethylether and analogue products, polymers, foam stabilizers; electrolytes and especially one or more thickeners. Thickeners that may be used in formulations of the present invention to assist in making the consistency of a product suitable include carbomer, siliciumdioxide, magnesium and/or aluminum silicates, beeswax, stearic acid, stearyl alcohol polysaccharides and their derivatives such as xanthan gum, hydroxypropyl cellulose, polyacrylamides, acrylate crosspolymers preferably a carbomer, such as carbopole® of type 980, 981, 1382, 2984, 5984 alone or mixtures thereof. Suitable neutralizing agents which may be included in the cosmetic formulations of the present invention to neutralize components such as e.g. an emulsifier or a foam builder/stabilizer include but are not limited to alkali hydroxides such as a sodium and potassium hydroxide; organic bases such as diethanolamine (DEA), triethanolamine (TEA), aminomethyl propanol, and mixtures thereof; amino acids such as arginine and lysine and any combination of any foregoing. The neutralizing agent can be present in an amount of about 0.01 wt. % to about 8 wt. % in the cosmetic formulation of the present invention, preferably, 1 wt. % to about 5 wt. %.

The addition of electrolytes into the cosmetic formulation of the present invention may be necessary to change the behavior of a hydrophobic emulsifier. Thus, the emulsions/microemulsions of this invention may contain preferably electrolytes of one or several salts including anions such as chloride, sulfates, carbonate, borate and aluminate, without being limited thereto. Other suitable electrolytes can be on the basis of organic anions such as, but not limited to, lactate, acetate, benzoate, propionate, tartrate and citrate. As cations preferably ammonium, alkylammonium, alkali- or alkaline earth metals, magnesium-, iron- or zinc-ions are selected. Especially preferred salts are potassium and sodium chloride, magnesium sulfate, zinc sulfate and mixtures thereof. Electrolytes can be present in an amount of about 0.01 wt. % to about 8 wt. % in the cosmetic formulations of the present invention.

The cosmetic formulations of the invention can preferably be provided in the form of a cream, milk, paste, stick, cleanser, balm, tonic, fluid, shampoo, hair spray, conditioner, mask, powder, enamel, enamel remover, solid tube stick, lipstick, foam, oil, soap, peeling, serum, ointment, gel, lotion, thickened lotion, and liquid. It can optionally be packaged as an aerosol and can be provided in the form of a mousse, foam or a spray. The cosmetic formulations according to the invention can also be in the form of a suspension or dispersion in solvents or fatty substances, or alternatively in the form of an emulsion or microemulsion (in particular of O/W or W/O type, O/W/O or W/O/W-type), such as a cream or a milk, a vesicular dispersion, in the form of an ointment, a gel, a solid tube stick or an aerosol mousse. The emulsions can also contain anionic, nonionic, cationic or amphoteric surfactants.

The topical application is preferably at least once per day, e.g. two or three times a day. Usually it takes at least two days until the desired effect is achieved. However, it can take several weeks or even months until the desired effect is achieved.

The amount of the cosmetic formulation which is to be applied to the skin depends on the concentration of the vitamin K₁ in the formulations and the desired cosmetic effect. For example, application can be such that a cream is applied to the skin. A cream is usually applied in an amount of 2 mg cream/cm² skin. The amount of the formulation which is applied to the skin is, however, not critical, and if with a certain amount of applied formulation the desired effect cannot be achieved, a higher concentration of the vitamin K₁ can be used e.g. by applying more of the formulation or by applying cosmetic formulations which contain more vitamin K₁.

According to the invention for preparing the formulation the vitamin K₁ can be used as such or in an encapsulated form, for example in a liposomal form. Liposomes are preferably formed with lecithins with or without addition of sterols or phytosterols. The encapsulation of the vitamin K₁ can be alone or together with other active ingredients, either together or in separate capsules.

Regarding the kind of the topical formulation and the preparation of the topical formulations as well as for further suitable additives, it can be referred to the pertinent literature, e.g. to Novak G. A., Die kosmetischen Präparate—Band 2, Die kosmetischen Präparate—Rezeptur, Rohstoffe, wissenschaftliche Grundlagen (Verlag für Chem. Industrie H. Ziolkowski K G, Augsburg).

It is furthermore possible to provide the cosmetic formulation of the present invention as oral formulation, e.g. in the form of pills, tablets, capsules which may contain granules or pellets, as a liquid, oral formulation or as an additive to foodstuff as is generally known to a skilled person. Useful procedures and useful additives for preparing the oral formulations of the present invention are e.g. disclosed in the standard literature Remington: The Science and Practice of Pharmacy, Lippincot, Williams & Wilking (Editors) 2000, which is included herein by reference.

As usual additives for oral formulations, in particular for tablets, usual excipients such as microcrystalline cellulose, sodium citrate, calcium carbonate, disodium or dipotassium phosphate, sodium or potassium phosphate, glycine, disintegration agents such as starch or alginic acid, binders such as polyvinylpyrolidone, saccharose, gelatin and gum arabicum lubricants such as magnesium stearate, sodium lauryl sulfate or talcum can be used. If the formulations are filled into gelatin capsules, usual additives for the preparation of granules are lactose or lactate as well as polyethylene glycols with a high molecular weight. Further additives and excipients as well as additives and excipients for other oral formulations and for food additives are known to a skilled person, and it can be referred to the pertinent literature such as “Grundzüge der Lebensmitteltechnik”, Horst Dieter Tscheuschner (Editor), 2. Edition, Hamburg, Beers 1996.

The content of the vitamin K₁ in the oral formulations of the present invention is usually about 1% to 90%, preferably about 10% to 80%, e.g. about 50% or more. The application is such that the desired effect occurs and depends on the human and the desired effect. A usual daily dosage can be in a range from about 0.1 μg/day to 50 mg/day, e.g. about 20 μg/day to 2 mg/day.

The following examples exemplify the invention, but they should not be construed as limiting the invention.

### EXAMPLE 1

The following cosmetic formulations were prepared:

| | | wt.-% | | | |
|---|---|---|---|---|---|
| | | | UV-protection | | |
| | ingredient | facial cream 1 | day care cream | facial cream 2 | body lotion |
| A) | Tegsoft Tn | 15.00 | 15.00 | 15.00 | 15.00 |
| | Estol GMM 3650 | 4.00 | 4.00 | — | — |
| | glyceryl myristate | — | — | 4.00 | 4.00 |
| | cethyl alcohol | 2.00 | 2.00 | 2.00 | 2.00 |
| | AMPHISOL K | 2.00 | 2.00 | 2.00 | 2.00 |
| | BHT | 0.05 | 0.05 | 0.05 | 0.05 |
| | Edeta BD | 0.10 | 0.10 | 0.10 | 0.10 |
| | Phenonip | 0.60 | 0.80 | 0.80 | 0.80 |
| | vitamin K₁ | 1.00 | — | — | — |
| | Parsol MCX | — | 5.00 | — | — |
| | Parsol 1789 | — | 2.00 | — | — |
| | Parsol 340 | — | 1.70 | — | — |
| B) | water | 67.94 | 60.55 | 66.45 | 70.15 |
| | propylene glycol | 5.00 | 5.00 | 5.00 | 5.00 |
| | Carbopol EDT 2001 | 0.30 | 0.30 | 0.30 | 0.30 |
| C) | vitamin K₁ | — | 1.00 | 1.00 | 0.10 |
| | STAY-C 50 | — | — | 1.00 | — |
| | water | — | — | 2.00 | — |
| D) | KOH 10% sol. | 2.01 | — | — | — |
| | KOH 50% sol. | — | 0.50 | 0.30 | 0.50 |
| | | 100.00 | 100.00 | 100.00 | 100.00 |

Procedures:

Facial cream 1: Heat part A and B separately to 80° C. Add part B to part A under stirring. Homogenize with an Ultra Turrax 30 seconds at 9500 rpm. Let cool down to room temperature under stirring and adjust the pH with part D.

UV-protection day care cream, facial cream 2, body lotion: Heat part A and B separately to 90° C. Add part B to part A under stirring. Homogenize with an Ultra Turrax 30 seconds at 9500 rpm. Let cool down to 40° C. under stirring and add part C. At room temperature adjust the pH with part D.

### EXAMPLE 2

To prove the energizing effect of vitamin K₁ human primary fibroblasts were chosen as an in vitro test system. The biological endpoint in the example is the energy level of each individual cell. This is determined quantitatively by the level of adenosine triphosphate (ATP), which is the common energy storage molecule in human cells.

a) Culture of Human Epidermal Fibroblasts

Primary epidermal fibroblast were isolated from human foreskin and cultured in DMEM with 10% fetal-bovine serum-(FBS), penicillin, and streptomycin. The cells were maintained in a growth chamber at 37° C. and 5% CO₂ and used for these kinds of experiments between passage 8 and 12. Fibroblasts were transferred to 96 well plates and allowed to attach for 5 h in standard medium before a serum starvation with 1% FBS over 4 days was initiated.

b) In Vitro Analysis of ATP Levels in Human Epidermal Fibroblasts

Epidermal fibroblasts were treated between 4 h and 48 h with vitamin K₁ at concentrations up to 44 μM in DMEM. After variing incubation time, the ATP-assay was performed with the High Sensitivy Cell Proliferation Assay (Via light Plus, Cambrex). The procedure was done according to the manufacturers recommendations. Briefly, the culture plate was removed from the incubator and was allowed to cool down to room temperature for at least 5 minutes, then 50 μl of Cell Lysis Reagent were added to each well and incubated 10 minutes, 100 μl of AMR PLUS were added to each well and incubated for 2 minutes at room temperature. The plate was analysed in a luminometer.

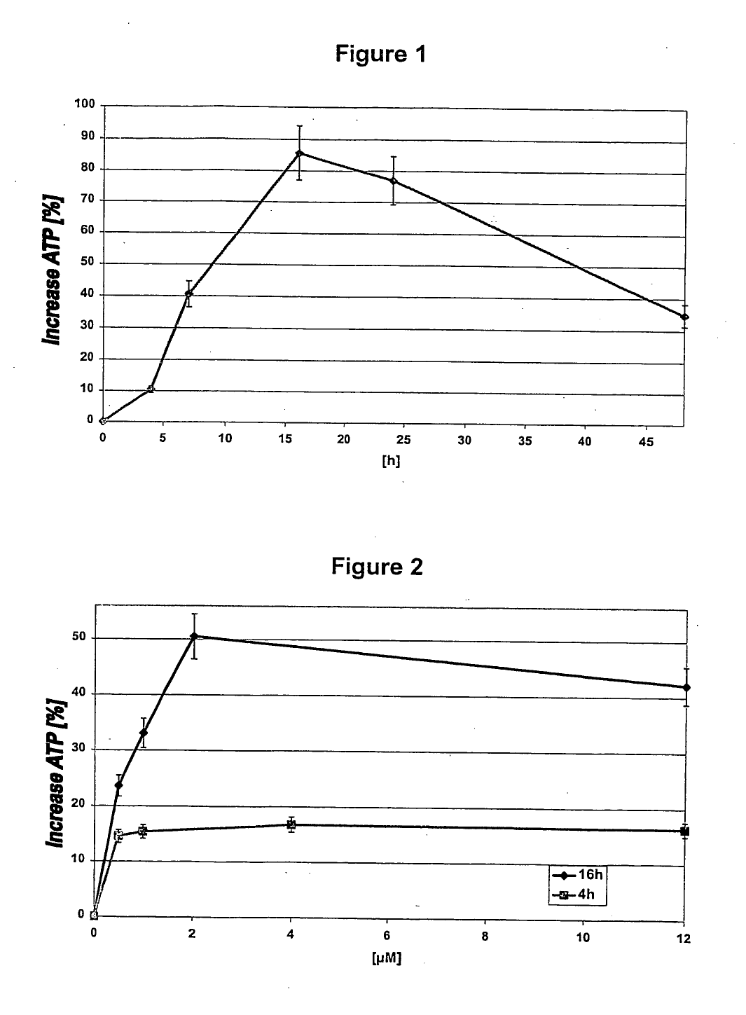

The results are illustrated in FIGS. 1 and 2. FIG. 1 shows the ATP levels in fibroblast with 2 μM vitamin K₁ in a time-dependent manner. Maximum stimulation is reached after an incubation time of about 16 h. FIG. 2 shows the ATP levels in fibroblast in a dose-response curve of vitamin K₁. Dose-response of vitamin K₁ is shown at two different time points. The most promising induction by vitamin K₁ is shown at a concentration of 2 μM which has no correlation to the time point.

1. A method to provide a stimulating, regenerating or conditioning effect on skin cells comprising applying to skin cells a cosmetic formulation which comprises vitamin K1 as an energizer.

2. Method according to claim 1, wherein vitamin K1 is contained in the cosmetic formulation in a concentration effective to provide a concentration of vitamin K1 in skin cells above 1 μM when the cosmetic formulation is applied thereto.

3. Method according to claim 1, wherein vitamin K₁ is contained in the cosmetic formulation in a concentration ranging from 0.001% to 5.0%.

4. Method according to claim 1, wherein the cosmetic formulation comprises vitamin K₁ in combination with a compound selected from the group consisting of vitamin C or a derivative of vitamin C, coenzyme Q₁₀ and an UV absorbing substance.

5. A topical cosmetic formulation containing vitamin K₁ and vitamin C or a derivative of vitamin C.

6. The formulation according to claim 5, characterized in that the derivative of vitamin C is ascorbyl phosphate or a cosmetically acceptable salt thereof.

7. A topical cosmetic formulation containing vitamin K₁ and an UV absorbing substance selected from the group consisting of
a polysiloxane-based UV-absorber,
1-(4-tert-butylphenyl)-3-(4-methoxyphenyl)-propane-1,3-dione,
2-cyano-3,3-diphenyl-acrylic acid 2-ethyl-hexyl ester,
(E)-rac-1,7,7-trimethyl-3-(4-methyl-benzylidene)-bicyclo[2.2.1]heptan-2-one,
2-phenyl-1H-benzimidazole-5-sulphonic acid, and
(E)-3-(4-methoxy-phenyl)-propionic acid 2-ethyl-hexyl ester.

8. A topical cosmetic formulation containing vitamin K₁ and coenzyme Q₁₀.

9. The formulation according to claim 8, characterized in that it contains vitamin C or a derivative of vitamin C.

10. The formulation according to claim 5, characterized in that it is selected from the group consisting of creams, pastes, sticks, cleansers, balms, tonics, fluids, shampoos, hair sprays, conditioners, masks, powders, enamels, enamel removers, lipsticks, foams, oils, soaps, peelings, serums, ointments, gels, lotions and liquids and facial tissues.
